(19) United States
(12) Patent Application Publication
(10) Pub. No.: US 2011/0230902 A1
(43) Pub. Date: Sep. 22, 2011

(54) ANASTOMOSIS DEVICE

(75) Inventor: Hassan Kansoul, Norsborg (SE)

(73) Assignee: Prozeo Vascular Implant AB

(21) Appl. No.: 13/129,471

(22) PCT Filed: Oct. 27, 2009
(86) PCT No.: PCT/SE09/51224

§ 371 Date: May 16, 2011

(30) Foreign Application Priority Data
Nov. 21, 2008 (SE) 0850084-5

(51) Int. Cl. A61B 17/11

(52) U.S. Cl. 606/153

(57) ABSTRACT

An anastomosis device including a graft vessel having a tubular body with two open ends. At least one of the ends is provided with a ring-shaped coupling unit integrated with the tubular body. The unit has a connection means element for coupling it to a complementary ring-shaped coupling unit. Anastomosis thereby can easily be made by connecting the graft vessel to a coupling unit attached on a target vessel, when coupling the two units together.

### FIELD OF INVENTION

The present invention relates to an anastomosis device including a graft vessel having a tubular portion with two open ends.

### BACKGROUND OF INVENTION

Vascular surgery often involves anastomosis between a blood vessel and a vascular graft to cerate blood supply to tissues. One important example is corona artery bypass surgery. Suturing the anastomosis is difficult and time-consuming. It requires the surgeon to be very experienced and skilful, and it is a large risk that the seal will be leaking. Therefore attempts have been made to find alternatives to conventional surgery for connecting a vascular graft to a blood vessel. Examples thereof can be found in U.S. Pat. No. 4,366,819, U.S. Pat. No. 6,171,321, U.S. Pat. No. 6,524,322 and U.S. 2004/0054405.

U.S. Pat. No. 4,366,819 discloses an anastomosis fitting for coronary artery bypass graft surgery having an assembly of four components including a cylindrical tube having at least one ringflange locking indentation in an inflow end and a plurality of locking ring grooves in an outflow end, a ringflange having a central aperture and pluralities of long and short spikes, the long spikes engaging in the locking indentation, with a graft engaged therebetween, a fixation ring having a central aperture and a plurality of spikes positioned about the aperture and a locking ring having an aperture with a plurality of locking ring ridges for engagement with the locking ring grooves. At surgical implantation an aortic wall having a hole therein engages between the ringflange and the fixation ring and is held in position by the spikes of the fixation ring, and the four components engage together forming an integral anastomosic fitting.

One disadvantage with this device is that it includes a plurality of components that the surgery has to fit together when connecting the graft to the blood vessel. Thereby a substantial surgical work still remains and the drawbacks of conventional surgery are only partly overcome.

U.S. Pat. No. 6,171,321 discloses a system for performing an end-to-side vascular anastomosis, including an anastomosis device, an application instrument and methods for performing an anastomosis between a vascular graft and the ascending aorta in coronary artery bypass surgery, particularly in port-access CABG surgery. A first aspect of the invention includes a vascular anastomosis staple. A first configuration has two parts: an anchor member, forming the attachment with the target vessel wall and a coupling member, forming the attachment with the bypass graft vessel. The anastomosis is completed by the coupling member, with the graft vessel attached, into the anchor member. A second configuration combines the function of the anchor member and the coupling member into one-piece anastomosis staple.

This device requires that the coupling member during the operation has to be attached to the graft vessel, which might be time consuming. Furthermore the attachment of the coupling member to the graft vessel requires large precision and skill from the surgeon in order to minimize the risk for leaking.

U.S. Pat. No. 6,524,322 discloses an anastomosis device for joining vessels, in the first place joining one end of a graft vessel to a target vessel at an opening made in the wall thereof. The anastomosis device comprises a tubular body on which an outer flange, which comes into contact with the outside of the wall of the target vessel around the opening, and an inner flange, which comes into contact with the inside of the wall of the target vessel around the opening, are arranged. The inner flange is made up of a number of arms which are able to move from an extended position, located in the extension of the tubular body, under the influence of a pre-tension into a position extending in the lateral direction with respect to the tubular body, after the pretension has been released, in order to form the inner flange.

Also this device includes the step of attaching the tubular coupling body to the graft vessel during the operation and thus entails drawbacks of similar kind as those relating to U.S. Pat. No. 6,171,321.

U.S. 2004/0054405 discloses a device for connecting two vascular prostheses, each having a tubular sidewall with a terminal portion extending to a connection end. A connection body has first and second terminal portions extending to first and second ends. The connector terminal portions are respectively mounted by the prostheses terminal portions. First and second straps respectively circumscribe the prostheses terminal portions to bias the prostheses into engagement with the connector body. The connector body has first and second strap engagement projections respectively captured by apertures in the straps.

The object of the present invention is to provide an anastomosis device for end to side anastomosis between a graft vessel and a natural vessel in the human body which reduces demands on time and skill of the surgeon as far as possible and overcomes the drawbacks related to prior art attempts in this respect.

### SUMMARY OF INVENTION

The object of the invention is achieved in that an anastomosis device of the kind initially specified includes the specific features that at least a first of said ends is provided with a ring-shaped coupling unit integrated with the tubular body and having connection means arranged for coupling to a complementary ring-shaped coupling unit.

The device radically reduces the complexity and time when joining a graft vessel to a natural blood vessel or the like in comparison with traditional surgery. Furthermore a minimum of pieces are required for the anastomosis, only two pieces are required, namely the graft vessel and an attachment device at the target vessel. The limited number of pieces required leads to a very secure and rapid operation, which makes the requirement on skill and experience of the surgeon less crucial. Since the coupling unit for attaching the graft vessel to the target vessel is an integrated part of the graft vessel itself, the need for attaching the graft vessel to a coupling unit is eliminated which further reduces the operation time and the risk for mistakes. As soon as a complementary coupling unit is attached to the target vessel only a simple plug-in step is required.

The device is particularly suitable for end-to-side anastomosis but it is to be understood that it also can be applied for end-to-end anastomosis where a graft vessel is involved.

With “complementary” is meant that the coupling units match each other such that they together forms a tight coupling, e.g. the coupling units are of male and female type, respectively.

According to a preferred embodiment also the second end of the graft vessel is provided with a second ring-shaped coupling unit integrated with the tubular body and having connection means arranged for coupling to a complementary ring-shaped coupling.

This embodiment allows a corresponding simple and safe connection at both ends of the graft vessel with a respective target vessel.

According to a further preferred embodiment the first and second coupling units are either identical or complementary.

This embodiment further simplifies the device and the operation when applying it, due to the uniform construction. Thus the attachment steps when attaching the ends of the graft vessel to a respective target vessel will be almost the same.

According to a further embodiment the tubular body includes at least two separate tubular sections and at least one interconnection device, each interconnection device having a first and second ring-shaped coupling part, each being integrated with a respective tubular unit, and the two ring-shaped coupling parts of the interconnection device being releasably interconnected.

The length required for the graft vessel is different for different kinds of applications in the human body and may also vary for the same kind of operation from one person to another. The length of the graft vessel thus is to be adapted to the actual condition. With a graft vessel built up by a plurality of sections according to this embodiment a very simple way of adapting the length is offered. The surgeon merely has to disengage the releasable interconnection device to have a shorter graft vessel, thereby any need to cut the graft vessel into the appropriate length is eliminated.

According to a further preferred embodiment the first ring-shaped part of the interconnection device is identical to the ring-shaped unit attached to said first end and in that the second ring-shaped part of the interconnection device is identical to the ring-shaped unit attached to said second end.

This embodiment has the advantage that if the surgeon needs to shorten the graft vessel by separating a portion thereof this portion will have its two ends identical to the complete graft vessel.

According to a further preferred embodiment the connection means of each ring-shaped coupling unit is either male connection means or female connection means, the male connection means including at least two axially extending members, each member having a free end, and the female connection means including at least two axially directed receiving openings, the axially directed receiving openings of a female connection means being arranges for axially receiving the free ends of the axially extending members of a male connection means, each axially extending member being arranged to be able to snap into a locking position in one of said receiving openings of a female connection means.

By providing the coupling means as a male or female coupling means in this way the connection of the graft vessel to the target vessel having a complementary coupling unit can be done very easy, rapid and with minimized risk for default. When the members have snapped into the receiving openings the axially locking eliminates the risk that the graft vessel will loosen from the coupling unit of the graft vessel.

According to a further preferred embodiment the graft vessel includes a further tubular part that is branched from the tubular body and connected thereto between its two ends and being in communication with the interior of the tubular body, which tubular part has an open end, which end is provided with a ring-shaped coupling unit integrated with the tubular part and having connection means arranged for coupling to a complementary ring-shaped unit. This embodiment is advantageous in applications where a graft vessel is to be connected to vessela in a Y-shaped configuration, e.g. for establishing communication between a patient's aorta and iliac arteries.

According to a further preferred embodiment of the invention an anastomosis system includes a graft vessel according to the invention, in particular to any of the preferred embodiments thereof, which system further includes a separate ring-shaped coupling unit having connection means that is complementary to the ring-shaped coupling unit of the graft vessel.

Thereby a complete system for the connection of a graft vessel to a target vessel is provided. Providing a system including both the components eliminates any risk for mismatch between the coupling unit of the graft vessel and the ring-shaped unit to which it is to be connected.

According to a further preferred embodiment the invented system includes that the separate ring-shaped coupling unit includes penetration means arranged to penetrate the wall of a vessel for attaching the separate ring-shaped coupling unit to the vessel.

This way of attaching the separate ring-shaped coupling unit is time-effective and allows a very secure attachment.

According to a further preferred embodiment the penetration means includes a vessel receiving surface including an area having a roughness with a large number of small sharp projections arranged to partly penetrate the wall of said vessel without reaching through said wall.

Since the projections do not completely reach through the wall they will not reach the inside of the connected vessel and there will be no opening facing the interior of the vessel that could cause infection or the like. With a large number of such small projections the connection will be sufficiently strong although each projection reaches only partly through the wall. By a large number in this application is meant between 50 and 10 000.

The above specified preferred embodiments are set out in the dependent claims.

The invention will be further explained by the following detailed description of examples thereof and with reference too the accompanying drawings.

### BRIEF DESCRIPTION OF THE DRAWINGS

FIG. 1 is a side view of a graft vessel according to the invention.

FIG. 2 is an end view of a separate coupling unit according to the invention.

FIG. 3 is an enlarged perspective view of a part of the graft vessel of FIG. 1.

FIG. 4 is an enlarged perspective view of the unit of FIG. 2.

FIG. 5 is an end view of FIG. 3.

FIG. 6 is an enlarged perspective view of a detail of FIG. 5.

FIG. 7 is an end view similar to that of FIG. 5 but showing an alternative example.

FIG. 8 is a perspective view of a part of the unit of FIG. 3 according to a still further example.

FIG. 9 is an enlarged section through details in FIGS. 3 and 4.

FIG. 10 is a section similar to that of FIG. 9 but illustrating an alternative example.

FIG. 11 is a side view of a graft vessel according to a further example of the invention.

FIG. 12 is a side view of a graft vessel according to still a further example of the invention.

FIG. 13 is a side view of a graft vessel according to still a further example of the invention.

### DETAILED DESCRIPTION OF EXAMPLES

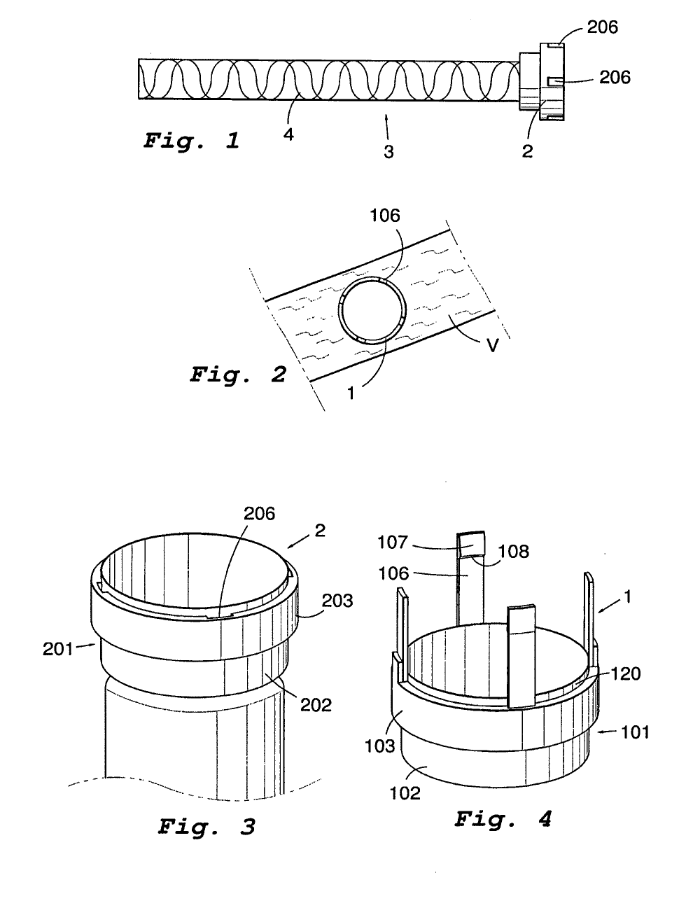

FIG. 1 in a side view illustrates a device according to a first example of the invention. The device includes a graft vessel 3 having a tubular body 4. The tubular body 4 of the graft vessel 3 is of conventional type and is made of reinforced PTFE, e.g. Dracon™ or Goretex™. At one of the ends the tubular body 4 has a s ring-shaped coupling unit 2 which is attached to the tubular body 4. The coupling unit 2 can be made of titanium or a suitable plastic, and is preferably glued or welded to the tubular body 4. Alternatively the coupling unit 2 is attached with some kind of fastening devices to the tubular body 4.

The coupling unit 1 is provided with four axially extending openings 206 on the side thereof facing away from the tubular body 4. The openings 206 are rranged to receive tongues from a complementary ring-shaped coupling as will be described later.

FIG. 2 illustrates a target vessel V to which the graft vessel is to be connected. A separate ring-shaped coupling unit 1 is attached to the target vessel V. The separate ring-shaped coupling unit 1 has four axially directed tongues 106 matching the openings 206 of the coupling unit 2 of the graft vessel.

When an anastomosis operation is to be made the surgeon makes an incision in the target vessel V and attaches the separate coupling unit 1 around the hole. Thereafter the graft vessel 3 is connected to the separate coupling unit 1 by snapping in the tongues 106 of the separate coupling unit 1 into the axial openings 206 of the coupling unit 2 of the graft vessel.

FIG. 3 is an enlarged perspective view of a part of the graft vessel 3 with the ring-shaped coupling unit 2. The coupling unit 2 has a body 201 that is generally cylindrical, and has a rear part 202 and a front part 203, where the front part 203 has slightly larger outer diameter than the rear part 202. In the illustrated embodiment the unit is circular cylindrical, but other cylindrical shapes, e.g. elliptical cylindrical are possible and in some instances even preferred from a functional physiological perspective.

At the edge of the front part 203a four axially extending openings 206 are provided. The number of openings can vary, but in most cases four openings is appropriate.

FIG. 4 is an enlarged perspective view of the separate coupling unit 1. Also this unit has a body 101 that is generally cylindrical, and has a rear part 102 and a front part 103, the front part 103 having a slightly larger outer diameter than the rear part 103.

Means for connecting the separate coupling unit 1 to the graft vessel coupling unit 2 extend substantially axially from the front edge of the front part 103. This means consists of axially extending tongues 106. The number of tongues 106 corresponds to the number of openings 206 in the graft vessel coupling unit 2, and the cross sectional shape of each tongue generally corresponds to the cross sectional shape of each opening 206.

The separate coupling unit 1 is to be attached to the target vessel V after a hole has been made in the wall of the target vessel V. Then the surgeon pulls the wall edges around the hole through the unit from its rear side until reaching the front side. Then the edges are folded radially outwards over a vessel receiving end surface 120 on the unit 1.

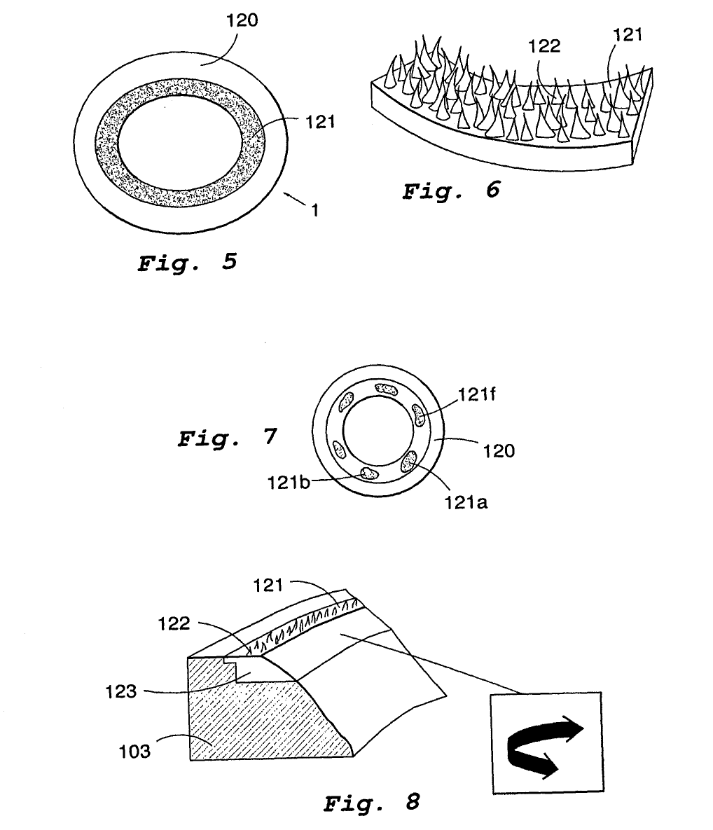

It is preferred to avoid a sharp edge between the hole and the end surface on which the vessel receiving end surface 120 is located. Therefore the cylindrical wall of the hole is connected to the end wall by a curved arc portion when seen in a section through the longitudinal axis of the unit. Preferably the curved arc portion is a circular arc portion. Thereby stress on the vessel is reduced and the risk of any cutting effect on the vessel wall is avoided. FIG. 5 schematically illustrates the vessel receiving surface 120 located on the axial end wall of the first attachment unit 1.

On the radially inner part of this surface 120 there is an area 121 that is rough, the roughness being created by a large number of short and sharp projections. In FIG. 6 these projections 122 are enlarged illustrated. The height of the projections 122 is in the range of 0,05-1,0 mm, preferably in the range of 0,1-0,5 mm.

When the walls of the target vessel around the hole is folded outwardly across the surface 120, the part of the vessel wall which thereby contacts the area 121 will be partly penetrated by the projections 122 such that the vessel is attached to the unit 1.

The height of the projections 122 in the prescribed range results in that the projections do not reach all the way through the vessel wall.

FIG. 7 illustrates an alternative example of arranging the vessel receiving surface 121. In this embodiment the area with the projections is constituted by a number of limited areas 121a to 121f.

FIG. 8 illustrates an example where the vessel receiving area 121 with the projections 122 is located on a separate ring member 123 which is rotatable in relation to the rest of the front part 103 of the separate coupling unit 1.

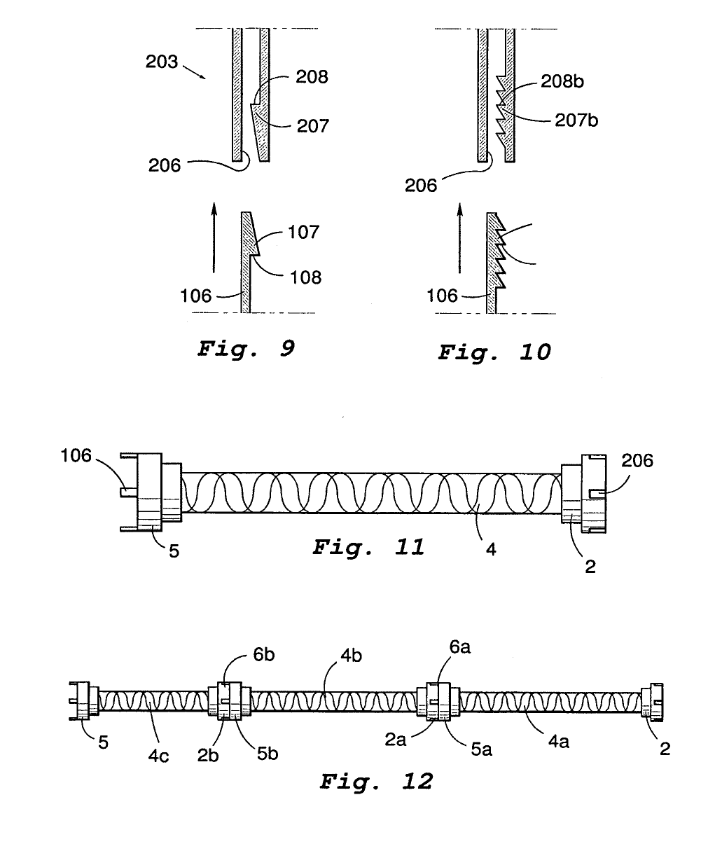

FIG. 9 is a schematic section through the end of one tongue 106 and one opening 206 and illustrates the snap-action and locking. As earlier described the end of tongue 106 has a radially offset part 107 forming a shoulder 108. One wall of the opening 206 in the second unit 2 is provided with a corresponding radially offset part 207 forming a shoulder 208.

When the end of the tongue 106 is inserted to the opening 206 as indicated by the arrow in the figure, the radially offset parts 107, 207 forces the end of the tongue 106 to be flexed to the left in the figure. When the shoulder 108 of the tongue 106 has passed the shoulder 208 of the opening 206 the end of tongue will flex back again and snap into a locking position where the shoulders 108, 208 abut against each other and lock the units against axial separating movement.

FIG. 10 illustrates in a corresponding section an alternative shape of the tongue 106 and the opening 206. In this case the tongue 106 at its outer end is serrated and thus has a saw-tooth profile 107b for cooperation with a corresponding saw-tooth-profile 207b on one wall of the opening. Thereby the tightness of the connection can be selected by the surgeon. Alternatively only either the tongue or the opening wall can be serrated and the other having solely one shoulder.

In the example illustrated in FIGS. 1 to 4, the coupling unit 2 of the graft vessel 3 is of female type and the separate coupling unit 1 is of male type. It is of course possible with a vice versa arrangement, i.e. the coupling unit 2 of the graft vessel 3 is provided with tongues and the separate coupling unit is provided with the axial openings.

It is also possible to arrange two tongues and two openings on the graft vessel coupling unit and correspondingly two openings and two tongues on the separate coupling unit, such that each unit has both female and male coupling members. In that case the two units can be identical with respect to the arrangement of the coupling members.

FIG. 11 illustrates an alternative example of the graft vessel 3. The right end thereof is similar to the example of FIG. 1. In this example however, also the other end has a coupling unit 5. The coupling unit 5 at that end is in the illustrated example of the male type. Alternatively both the coupling units 5 of the graft vessel can be of the same type, either male or female. The coupling unit 5 is arranged for connection to a second separate coupling unit that has been attached to a vessel in the body in a manner similar to that described in connection with FIG. 2.

FIG. 12 illustrates a further alternative example of the invented graft vessel. In this case the graft vessel consists of three separate tubular sections 4a, 4b, 4c. Like in the example of FIG. 11 the graft vessel has a coupling unit 2, 5 at each end thereof. The sections are connected by two interconnections devices 6a consist of two ring-shaped parts 2a, 5a attached to a respective tubular section 4b, 4a.

The ring-shaped parts 2a, 5a of the interconnection device are similar to the ring-shaped coupling units 2 and 5, respectively at the ends of the graft vessel 3. The two parts of the interconnection device thus are coupled to each other in a male-female relationship and can be released from each other. The surgeon thus can release either of the connection devices 6a, 6b in order to obtain a length of the graft vessel that corresponds to the actual need. It is to be understood that the number of sections not necessarily is three. The graft vessel can be constituted by two sections or more than three sections.

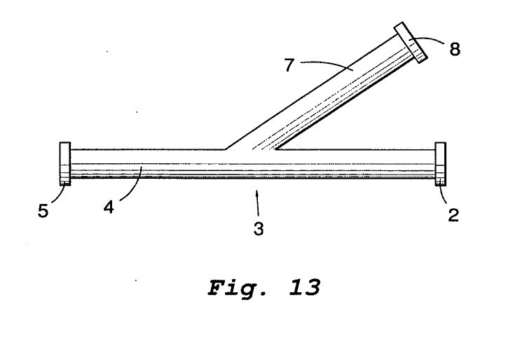

FIG. 13 illustrates an application where the graft vessel is to be attached at one end to one vessel and at the other end to two vessels. In this case the tubular body 4 has a branched tubular part 7, which also has a ring shaped coupling unit 8 at its free end.

Above has been described an operation where one end of the graft vessel is attached to the target vessel in an end-to-side anastomosis. It is to be understood that the invented device can be used for end-to-end anastomosis as well.

Although the invented device is particularly useful for corona artery bypass graft surgery it is to be understood that it can be applied also for other kind of anastomosis of blood vessels as well as for connecting other vessels in the body together.

1. An anastomosis device including a graft vessel having a tubular body with two open ends wherein at least a first of said ends is provided with a ring-shaped coupling unit integrated with the tubular body and having a connection element arranged for coupling to a complementary ring-shaped coupling unit.

2. The device according to claim 1, wherein the second end is provided with a second ring-shaped coupling unit integrated with the tubular body and having another connection element arranged for coupling to a complementary ring-shaped coupling unit.

3. The device according to claim 2, wherein the ring-shaped coupling units at the first and second ends are either identical or complementary.

4. The device according to claim 2, wherein the tubular body includes at least two separate tubular sections and at least one interconnection device, each interconnection device having a first and second ring-shaped coupling part, each being integrated with a respective tubular section and the two ring-shaped coupling parts being releasable interconnected.

5. The device according to claim 4, wherein the first ring-shaped part of the interconnection device is identical to the ring-shaped unit attached to said first end and in that the second ring-shaped unit of the interconnection device is identical to the ring-shaped unit attached to said second end.

6. The device according to claim 2, wherein the connection element of each ring-shaped coupling unit is either a male connection element or a female connection element, the male connection element including at least two axially extending members, each member having a fee end, and the female connection element including at least two axially directed receiving openings, the axially directed receiving openings of the female connection element being arranged for axially receiving the free ends of the axially extending members of the male connection element, each axially extending member being arranged to be able to snap into a locking position in one of said receiving openings of the female connection element.

7. The device according to claim 1, wherein the graft vessel includes a further tubular part that is branched from said tubular body and connected thereto between said two ends and is in communication with the interior of the tubular body, said tubular part having an open end, which end is provided with a ring-shaped coupling unit integrated with the tubular part and having another connection element arranged for coupling to a complementary ring-shaped unit.

8. An anastomosis system including a graft vessel according to claim 1, and a separate ring-shaped coupling unit, the separate ring-shaped coupling unit having a connection element that is complementary to the connection element of the ring-shaped coupling unit of the graft vessel.

9. The anastomosis system according to claim 8, wherein the separate ring-shaped coupling unit includes a penetration element arranged to penetrate the wall of a vessel for attaching the separate ring-shaped unit to the vessel.

10. The system according to claim 9, wherein the penetration element includes a vessel receiving surface including an area having a roughness with a large number of small sharp projections arranged to partly penetrate the wall of said vessel without reaching through said wall.

11. A method for anastomosis vessels in human or an animal comprising the steps of providing a graft vessel with a first end having a ring-shaped coupling unit having a connection element, providing a separate ring-shaped coupling unit, having another connection element that is complementary to the connection element of the ring-shaped coupling unit of the graft vessel,
attaching a second end of said graft vessel to a first vessel,
attaching the separate ring-shaped coupling unit to a second vessel, or to a second place of the first vessel, and
coupling together the ring-shaped coupling unit of the graft vessel and the separate ring-shaped coupling unit.

12. The method according to claim 11, wherein the graft vessel is provided with a second ring-shaped coupling unit attached to the second end thereof, which second ring-shaped coupling unit has another connection element arranged for coupling to a complementary ring-shaped coupling, and wherein the step of attaching the second end of said graft vessel to the first vessel includes the substeps of:
attaching a second separate ring-shaped coupling unit to said first vessel, and coupling together the second ring-shaped coupling unit of the graft vessel and the second separate ring-shaped coupling.

13. The method according to claim 12 wherein the provided graft vessel includes at least two separate tubular sections and at least one interconnection device, each interconnection device having a first and second ring-shaped coupling part, each being integrated with a respective tubular section and the two ring-shaped coupling parts being releasable interconnected, and wherein the method comprises the steps of:
releasing one of said interconnection parts thereby separating the graft vessel into two shorter graft vessels, and
utilizing one of said shorter graft vessels as the graft vessel for the anastomosis.

14. A method for anastomosis vessels in human or an animal wherein the method is performed by using a system according to claim 8, and wherein the method comprises the steps of:
attaching the second end of said graft vessel to a first vessel,
attaching the separate ring-shaped coupling unit to a second vessel, or to a second place of the first vessel, and
coupling together the ring-shaped coupling unit of the graft vessel and the separate ring-shaped coupling unit.

15. A method for anastomosis vessels in human or an animal wherein the method is performed by using a system according to claim 9, and wherein the method comprises the steps of:
attaching the second end of said graft vessel to a first vessel,
attaching the separate ring-shaped coupling unit to a second vessel, or to a second place of the first vessel, and
coupling together the ring-shaped coupling unit of the graft vessel and the separate ring-shaped coupling unit.

16. A method for anastomosis vessels in human or an animal wherein the method is performed by using a system according to claim 10, and wherein the method comprises the steps of:
attaching the second end of said graft vessel to a first vessel,
attaching the separate ring-shaped coupling unit to a second vessel, or to a second place of the first vessel, and
coupling together the ring-shaped coupling unit of the graft vessel and the separate ring-shaped coupling unit.
